(19) United States
(12) Patent Application Publication
(10) Pub. No.: US 2018/0353080 A1
(43) Pub. Date: Dec. 13, 2018

(54) TIME RESOLVED NEAR INFRARED REMISSION SPECTROSCOPY FOR NONINVASIVE IN VIVO BLOOD AND TISSUE ANALYSIS

(71) Applicants: Joseph Chaiken, Fayetteville, NY (US); Jerry Goodisman, Syracuse, NY (US)

(72) Inventors: Joseph Chaiken, Fayetteville, NY (US); Jerry Goodisman, Syracuse, NY (US)

(73) Assignee: Syracuse University, Syracuse, NY (US)

(21) Appl. No.: 15/781,720

(22) PCT Filed: Dec. 7, 2016
(86) PCT No.: PCT/US16/65319

§ 371 Date: Jun. 6, 2018

Related U.S. Application Data
(60) Provisional application No. 62/263,813, filed on Dec. 7, 2015.

(51) Int. Cl. A61B 5/00; A61B 5/145; A61B 5/1455

(52) U.S. Cl. CPC A61B 5/0075; A61B 5/4875; A61B 5/14556; A61B 5/14535

(57) ABSTRACT

A system and method for obtaining the intravascular plasma volume, red blood cell volume, oxygen saturation SpO2 and Hgb hemoglobin concentration from a sample of in vivo tissue. A sample is irradiated with pulses of single incident wavelength light on a sample of tissue. The prompt emission (PE) and the delayed (DE) light emitted from the tissue are measured simultaneously. A relative volume of light emitted from two phases contained within the tissue is then determined, wherein the two phases comprise a first Rayleigh and Mie scattering and fluorescent phase associated with red blood cells, and a second, non-scattering phase associated with plasma. The plasma volume, Hct, Hgb and SpO2 is calculated from the relative volume of light emitted by the first phase and the relative volume of light emitted from the second phase differentiated by state of oxygenation.

### CROSS-REFERENCE TO RELATED APPLICATIONS

The present application claims priority to U.S. Provisional No. 62/263,813, filed on Dec. 7, 2016.

### BACKGROUND OF THE INVENTION

### 1. Field of the Invention

The present invention relates to noninvasive analysis of blood and tissue in vivo and, more particularly, to a time resolved approach for near infrared remission spectroscopy.

### 2. Description of the Related Art

For a wide variety of medical and fitness reasons, there is a continuing need to monitor the relative state of hydration of tissues that are in contact with blood. For example, vital signs are indispensable indicators of health of a person. Temperature, pulse rate, breathing rate and blood pressure are the first quantitative and objective pieces of information that doctors rely upon at the outset of understanding the current state of their patients' health. For example, undetected internal bleeding is the leading preventable cause of all death in the sense that if the bleeding can be detected and located quickly enough, the patient can almost always be saved. And if not, the patient is almost always lost.

As monitoring must be noninvasive and highly portable to enable use in physically demanding situations, there is a preference for optical based approaches. Existing techniques, including those previously developed by the inventor of the present invention, can provide the necessary information but require systems having greater complexity, diminished reliability, and higher costs.

### BRIEF SUMMARY OF THE INVENTION

The present invention involves the excitation of perfused tissue with a pulse of a single wavelength near infrared (NIR) light. All of the light is collected from a point proximate to its entry location. Light that comes out first, i.e., the light with the shortest time delay, was elastically and inelastically scattered inside the tissues. Inelastically scattered prompt emission is very weak, however, compared to the elastically scattered light and thus can be ignored. The time-delayed light is nearly all inelastically scattered and can be treated as a single signal. A clean separation between elastic and inelastic signals emanating from the blood and the static tissues can be achieved by monitoring the time resolved optical response when NIR light is directed into composite tissues. This signal can then be analyzed using radiation transfer theory, keeping only linear terms, to obtain hematocrit and plasma volume.

The present technology requires only a single wavelength of light to be introduced into the tissue, a minimum of no filters, and only a single photodetector that is analyzed in a time resolved manner. The approach of the present invention is robust in compensating for photobleaching effects that limit conventional spectral response approaches for short times at the beginning of monitoring before steady state can be achieved. The present approach can also be exploited to separate Raman scattered light from fluorescence from NIR excited tissues.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of a system for time resolved near infrared remission spectroscopy for noninvasive in vivo blood and tissue analysis; and

FIG. 2 is a graph of prompt emission (PE) light and delayed emission (DE) light used to estimate red blood cell and plasma levels of a sample;

FIG. 3 is a graph of the spectrum of remitted light when in vivo tissue is probed with CW 830 nm laser light;

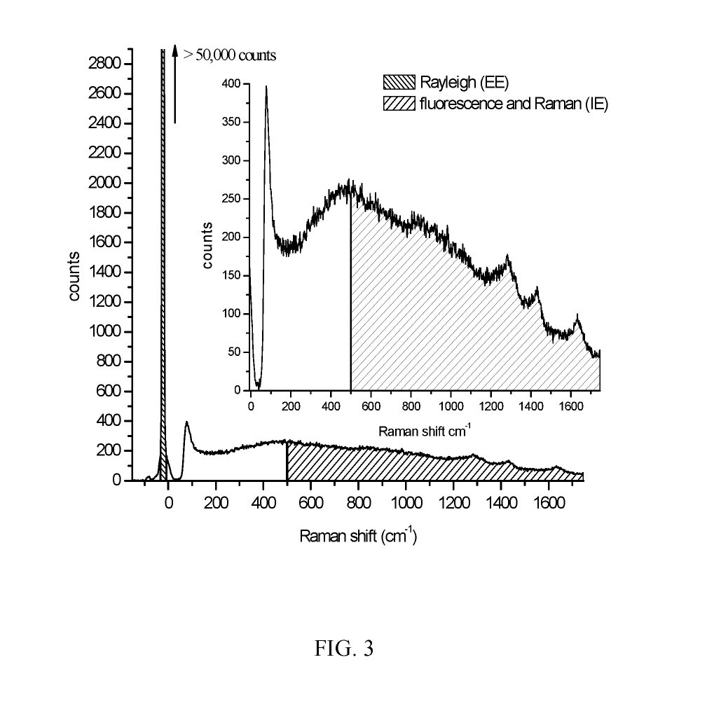

FIG. 4 is a graph of the absorption spectra of oxy and deoxy hemoglobin in the near infrared (NIR) spectral range where the PE for 830 nm excitation is indicated by the patterned section and the isosbestic point is the wavelength where the absorption per molecule is the same for both oxy and deoxy hemoglobin;

FIG. 5 is a graph of apparent Hct calculated using the approach of the present invention where time is given in hemocycles (hc) with 1 hc=3 seconds and Hct was calibrated against the CritLine using data from a different test subject at constant SPO₂.

### DETAILED DESCRIPTION OF THE INVENTION

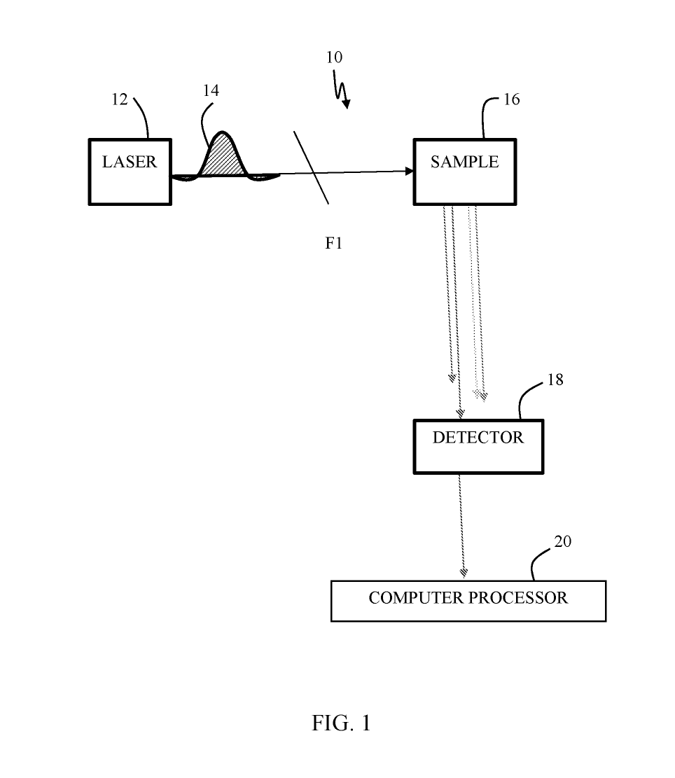

Referring now to the drawings, wherein like reference numerals refer to like parts throughout, there is seen in FIG. 1 a system 10 for time resolved near infrared remission spectroscopy for noninvasive in vivo blood and tissue analysis. System 10 comprises a light source 12, such as a laser, for providing a pulsed input 14 to a sample 16. The multi-wavelength output 18 is collected by a single channel detector 20 that provides data representing the measurement of prompt emission (PE) light and delayed emission (DE) light to a processor 22. A filter F may be used to attenuate incident light from a laser to remove low intensity wings.

Processor 22 may be programmed to determine the intravascular plasma volume and red blood cell volume when a sample of in vivo tissue is irradiated by system 10. In general, processor 22 is programmed to determine the relative volume of light emitted from two phases contained within the tissue, wherein the two phases comprise a first Rayleigh and Mie scattering and fluorescent phase associated with red blood cells, and a second, non-scattering phase associated with plasma. The plasma volume is calculated from the relative volume of light emitted by the first phase and the relative volume of light emitted from the second phase.

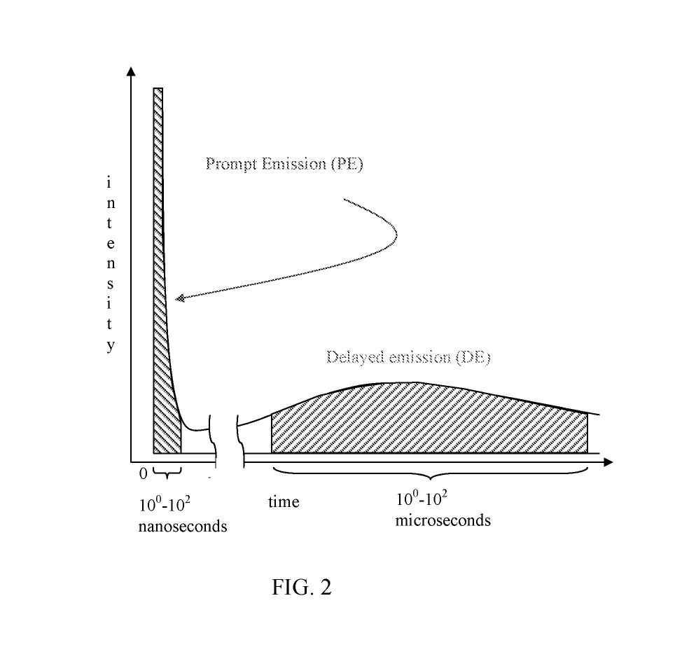

In a typical embodiment, the incident wavelength is anything between 580 and 2500 nm (for in vitro situations the incident light can be from 157 nm and extend up to 2500 nm). In preferred embodiments, the incident wavelength may be 785, 805 or 830 nm. The time delay associated with the PE measuring is typically in a window of time starting with the leading edge of the incident light pulse to within 20 nanoseconds of the end of the incident light pulse (or edge in the case of chopped light), and the DE over the interval that starts from 300 nanoseconds after the end of the incident pulse to 100 microseconds after the end of the light pulse. FIG. 2 illustrates this time delay and the signals received by detector 18.

In one embodiment, system 10 determines the relative plasma volume as follows:

$$\begin{matrix}{φ}_{p} / ( {φ}_{r} + {φ}_{p} ) & [ 1 ] \\ PE = {C}_{1} + {C}_{2} {φ}_{p} + {C}_{3} {φ}_{r} & [ 2 ] \\ DE = {C}_{4} + {C}_{5} {φ}_{p} + {C}_{6} {φ}_{r} & [ 3 ] \\ {φ}_{r} = a + b ( \frac{PE}{{PE}_{0}} ) + c ( \frac{DE}{{DE}_{0}} ) \\ wherein & [ 4 ] \\ {φ}_{p} = d + e ( \frac{PE}{{PE}_{0}} ) + f ( \frac{DE}{{DE}_{0}} ) & [ 5 ]\end{matrix}$$

wherein: PE is total promptly emitted light, DE is total delayed emitted light, C₁ and C₄ are the fractions of PE and DE, respectively, from static tissue; C₂ and C₅ are the fractions of PE and DE, respectively, from plasma; C₃ and C₆ are the fractions of PE and DE, respectively, from red blood cells; C₁₋₆ can be calculated numerically using the radiative transport equation (RTE) using optical and geometric parameters appropriate to the tissues and instrumentation appropriate to the specific probing, to determine PE and DE as a function of ϕᵣ and ϕₚ; PEₒ and DEₒ are calculated or measured average values of PE and DE over a calibration time period that depends on the laser power and volume of tissue probed under a reference condition.

Values for a-f can be obtained by inverting equations [2] and [3] to express ϕᵣ and ϕₚ in terms of PE and DE, or if fluctuations from homeostasis are the desired measurement, with the result being of the form [4] and [5].

In another method, one could calculate the plasma volume from equation 1 and compare that to measurement on actual human subjects who are undergoing dialysis. For example, Gambro (Fresenius) dialysis machines utilize a device called the CritLine that measures the hematocrit and the associated plasma volume in real time using the blood inside the dialysis machine. Raw DE and PE can be measured in real time while the dialysis is occurring. Afterwards, the system of equations is over sampled and a-f can be calculated using any of several commercially available mathematical analysis programs such as Excel Solver.

A full set of optimized a-f parameters so obtained can be used later for the same person or different people to monitor any changes of the hematocrit in time.

In a typical embodiment, the tissue is human. Other species, particularly primates and other vertebrates and invertebrates, can also be subjects for whom the method is useful. Typically, the tissue is a fingertip, although those skilled in the art will appreciate the applicability of the method to other areas of the body. In one embodiment, the fingertip is pressed against an aperture of an apparatus that emits light directed at the fingertip through the aperture. In a typical embodiment, the pressure at which the fingertip is initially pressed is approximately the average of the prevailing systolic and diastolic blood pressures of the subject or the Mean Arterial Pressure (MAP).

In an embodiment of the invention, the laser pulse duration may be <400 nanoseconds, with <100 nanoseconds preferred, with a repetition rate of 5-10 kHz and an average power @ 10kHz=20 μW<P_{10 kHz}<500 mW (with a preferred range of 100 mW<P_{10 kHz}<200 mW). Polarized or unpolarized light may be used.

For determination of PE parameters, all remitted light between initial 1/e point (37% of eventual maximum) may be integrated with dropping of the 1/e point (falling and at 37% of maximum). For DE parameters, all remitted light is integrated starting when total remitted light begins rising after short time minimum, as per break in FIG. 2 time scale. Alternatively, a specific “integration start point” may be selected, such as the earliest time when remitted light is essentially zero, i.e., after a light pulse is first directed on non-fluorescent target. Alternatively, it is possible to specify a DE starting point between 300 nanoseconds after beginning of pulse to as long as 10 microseconds after same initial leading edge of incident pulse.

The present invention may be used to determine the measure of the hematocrit of the blood in the capillaries and the measurement of the volume of plasma in the capillaries. Hematocrit, i.e., the percentage by volume of the blood that is red blood cells, has long been known to reflect the thickness, i.e., the viscosity, of the blood. The plasma volume reflects the total amount of liquid inside the capillaries and has not been readily accessible to doctors before. Knowing how these two numbers change, with unprecedented accuracy and precision, provides the earliest indications of internal bleeding even when there is no external injury. There are many additional applications of the capacity to monitor the balance between intravascular fluids and extra-vascular fluids such that the stability of the two parameters provided by the present invention device constitutes a single new vital sign. The device in operation is much like the ubiquitous pulse oximeter with a clip on one finger, painless and benign. Other locations can be monitored and the PVH and pulse oximeter could be integrated into a single clip.

As the medical community becomes more aware of the information provided, the present invention may be included in EMT vehicles and in patient monitors in hospital rooms. Patients may be monitored after all surgeries, from routine to serious, to ensure that there is no bleeding afterwards. From multiple myeloma to ulcers and Crohn's Disease, the ability to detect even slow internal bleeding or compartment shifts of fluids allows clinicians more clear courses of action. The ability to detect at a very early stage, internal fluid shifts that occur for various reasons, but that all lead to swollen hands, legs, feet and other body parts, will allow more successful interventions and the avoidance of further complications.

For fitness and readiness applications and products, the present invention can allow people to assess their own hydration state and make changes as they prefer. For assessing military readiness and fitness, the present invention can used to collect data that is report remotely by RF or Bluetooth to provide real-time assessments of fitness and timely assignment of personnel.

The present invention may also be used for the measurement of blood oxygenation. The light produced within the probed volume must traverse tissue before it can be collected outside the tissue. The fluorescence is produced by hemoglobin and other materials in the plasma and static tissues. The amount of fluorescence produced per molecule by hemoglobin with 830 nm excitation is much less than that produced by 785 nm excitation. Using 830 nm excitation the majority of the DE is from the static tissue and the plasma. Since they are produced by physically independent processes, the DE and the PE can be distinguished from each other in a temporal sense, used pulsed probing light. PE light experiences no delay in that can be detected as the first light that exits the tissue after the probing pulse enters. DE, on the other hand, is created from a sequence of more complicated processes involving the probing light first being absorbed by molecules in the probed volume i.e. static tissue, the RBCs and the plasma then the conversion of that energy into other kinds of molecular motion followed by emission of lower energy photons and so it is necessarily delayed. So if the probing light consists of a short pulse or even a train of sufficiently short pulses, DE can be discerned from PE by the temporal delay. It must be noted that Raman scattering does not have this delay but fluorescence, which comprises greater than 99 percent of the total DE remitted light, does have the delay. In the temporal sense, the Raman scattered remitted light is partitioned to the PE. Since the Raman scattering from tissue is very weak compared to either the fluorescence or the elastically scattered light, it can be ignored for the present invention.

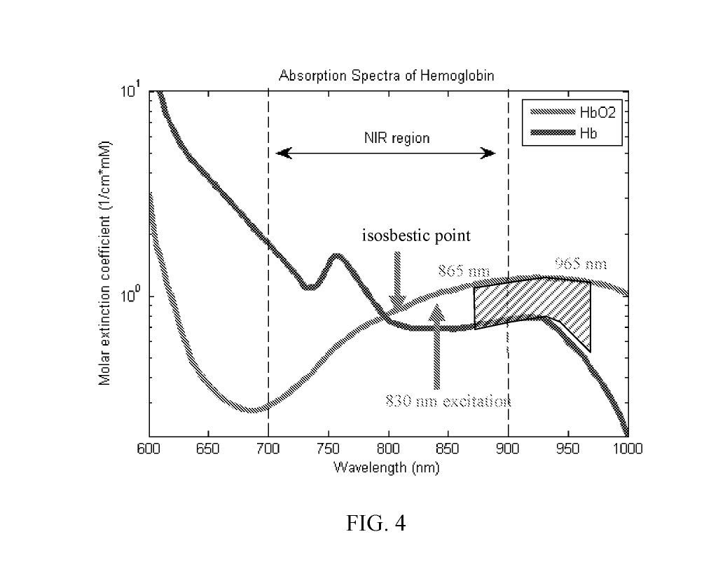

The scattering properties of red blood cells containing oxyhemoglobin are about the same as for those containing deoxyhemoglobin so the net effect of variable blood oxygen saturation (SpO₂) on the output of the algorithm of the present invention is due to variable absorption of the fluorescence in the probed volume before it can be remitted and detected, as seen in FIG. 4.

If the DE is chosen, FIG. 4 shows that variable SpO₂ will modulate the amount of DE collected. If the excitation wavelength is chosen such the DE overlaps the isosbestic point, then (depending on the exact wavelengths involved) the modulation effect will be much less because the absorption at the wavelength of the isosbestic point itself is independent of SpO₂ and the absorption on either side of the isosbestic point will tend to self-compensate, i.e., more absorption on one side will tend to compensate for less on the other side leading to low net sensitivity to SpO₂. Note that if the DE doesn't overlap the isosbestic point, then a net absorption occurs and variable SpO₂ will modulate the “apparent” hematocrit (Hct) and PV. This means that it is possible by judicious choice of a single excitation wavelength to simultaneously monitor changes in Hct, PV and SpO₂ noninvasively and in vivo. If one chooses to utilize the isosbestic point appropriately, one can use the present invention in a manner that removes the SPO₂ sensitivity.

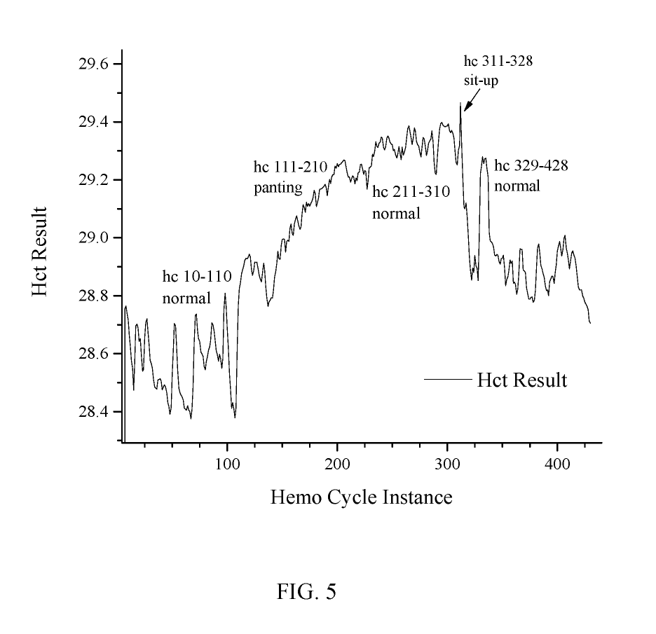

An example of this is shown in FIG. 5. In this case, a fiber coupled probe is attached to the volar side of one big toe with the test subject in a supine position. The first 110 hc correspond to a resting breathing and pulse rate. When the subject began to pant at 111 hc the oxygenation increased from 95% to 98% as indicated independently using a pulse oximeter. At 211 hc the subject returned to normal breathing and the oxygenation began to level out but did not decrease as confirmed by pulse oximetry. Starting at 311 hc the subject began doing sit ups, and consistent with physical exercise recruiting blood for the muscular exertion and the increased oxygen demand, the apparent Hct decreased. When the sit ups were stopped at hc 328, the recruiting stopped and the oxygen demand returned to a resting level. The apparent Hct first rebounded due to the restored peripheral perfusion and the localized presence of residual oxygen. As the resting state was extended, the apparent Hct tended towards its original level. It does not return to the original level because throughout the demonstration the test subject experienced intravascular fluid loss due to insensible perspiration and kidney action.

Clearly, using appropriate filtering one could use two sections of the DE, one at the isosbestic point (DEᵢₛₒ) and the other closer to the water absorption (DE_{H2O}) in order to calculate the total hemoglobin concentration (Hgb). In this case the ratio of the remitted intensity at the two wavelengths i.e. DEᵢₛₒ/DE_{H2O} would be proportional to the Hgb. It must be emphasized that physiologically Hgb and Hct are two different quantities. Hgb relates more to the oxygen carrying capacity of the blood since it originates with the hemoglobin molecules whereas the Hct relates more to the viscosity of the blood since it relies on the RBCs themselves. Variation of these two quantities has different interpretations clinically.

Note that once a device according to the present invention has been calibrated, such as by using a CritLine, comparison of the “apparent Hct” under conditions of known constant Hct, but variable SpO, to an independent pulse oximeter can calibrate the “apparent Hct” to the SpO₂ changes.

There are many uses for a device that has superior sensitivity, accuracy and precision than any two existing devices capable of obtaining the same information. This device can be made very small and wirelessly connected with a very small power requirement

Commercial embodiments of the present invention may use pulsed lasers that are available off-the-shelf and may include packaging with a “clean-up filter.” This filter limits the wavelength range of the raw laser spectrum because lasers are not necessarily single wavelength devices and often emit a narrow range of wavelengths such that even at a short shift from the center wavelength there is sufficient incident light to swamp most DE signals.

The tissue of a target subject must be positioned relative to the laser in a manner that is stationary while not constricting the tissue in any manner such that the blood flow will be interrupted excessively. The tissue begins to “bleach” as soon as light impinges on it. In the present invention, a certain level of DE and PE is collected for each pulse of laser light that probes the tissue. “Bleaching” means the amount of DE produced decreases in time, i.e., successive pulses decrease until a stable level is reached and does not vary in a monotonic manner with each pulse. This bleaching constitutes a decrease in the autofluorescence produced by nearly all fresh biological materials. If the tissue is a physically stable position relative to the incoming laser light, the same tissue will be probed by each successive pulse, and the stable DE will be reached. If the tissue is not in a physically stable position relative to the incoming laser light, then unbleached tissue will be contacted by different pulses and the stable pulse-to-pulse DE will not be attained.

Any physical contact between an external solid surface and perfused tissue will nearly always affect blood flow within the tissue, (except for bone, of course) and the affect is to causes fluctuations in the quantities of interest, such as Hct and plasma volume localized in the probed volume. The force or pressure used to ensure stationary placement must not exceed the local systolic blood pressure or there is restricted blood movement. To be stationary it must exceed the diastolic pressure. Spring loaded clips, such as those common in SpO₂ and Hgb devices, may be used. In addition, probes embodying the present invention may be provided with flat or other shaped surfaces for use at various locations on the body. These probes can be fastened or otherwise held in place by adhesives or Velcro straps.

The shape of the surface in actual contact with the skin or other tissue is also important. The contact produces a stress field and a perfectly flat surface making contact with the target tissue may produce an underlying blood movement that is less steady than if there is some definite shape to the points of contact between the surface and the tissue. For example, an aperture through which the light passes to define the contact between the shape of the hole and skin may be used.

An embodiment of the present invention may thus use an aperture with a predetermined diameter ranging from 2 mm to 10 mm with focusing optics on the other side of the hole of about f2 (NA=0.5) that can be downgraded to f3 depending on the thickness of the tissue being probed. The idea is to only sample light being remitted from tissue perfused by capillaries. The thickness of the material making contact with the skin surface should be chosen such that given the focal length of the last optic and the dimension of the aperture, the light is focused in the perfused tissue closest to the surface with an acceptable f number or numerical aperture (NA) specified above. With this design the last optic can be placed in contact with the other side of the material comprising the contact surface. This simultaneously insures that the skin and optics are maintained in proper positioning for the reasons stated above and the inside of the device nearest the tissue is sealed so that materials cannot enter from the outside.

To measure analyte SpO₂ or HgbO₂/(Hgb) an excitation wavelength of 805-850 nanometers (805-810 nanometers preferred) may be used, such as that seen in FIG. 4. To measure analyte Hgb independent of oxy/deoxy, an excitation wavelength in the range 750-790 nanometers (with 785 preferred) may be used. To measure analyte hematocrit (Hct) and/or plasma volume (percent of total blood volume occupied by plasma), an excitation wavelength in the range of 800-830 nanometers (790-810 nanometers preferred) may be selected.

To unambiguously produce four separate analytes simultaneously Hct, PV, SpO2, Hgb, the present invention may operate at two different excitation wavelengths simultaneously with the use of Principle Component Analysis (PCA) to assign a value to each analyte in terms of the four independent wavelengths, i.e., the PE and DE for each of the two wavelengths. The two different wavelengths are chosen so that one is in the range of 750-790 nm (785 nm preferred), and the other is either 805 to 850 nm (805-810 nm preferred) or 800 to 830 nm (790-810 nm preferred). Alternatively, the Partial Least Squares (PLS) approach may used to correlate Hct, PV, SpO₂, Hgb obtained using the present invention with independent measurements of the analytes using conventional technology, such as of Hct (CritLine), PV (CritLine), SpO2 (Masimo, Welch Allyn, Nellcore), Hgb (Masimo, Welch Allyn, Nellcore) at each time point. Other chemometric analyses can also be used but PLS or PCA are preferred.

What is claimed is:

1. A device for in vivo blood and tissue analysis, comprising:
a light source positioned to illuminate an in vivo tissue sample with at least one pulse of near infrared light;
a single channel detector positioned to collect any multi-wavelength light emitted from the in vivo tissue sample in response to the series of pulses of light;
a processor programmed to determine the amount of prompt emission light and the amount delayed emission light in the multi-wavelength light emitted from the in vivo tissue sample.

2. The device of claim 1, wherein the processor is further programmed to calculate an intravascular plasma volume and a red blood cell volume of the in vivo tissue sample based on the amount of prompt emission light and the amount of delayed emission light.

3. The device of claim 2, wherein the processor is programmed to calculate an intravascular plasma volume and a red blood cell volume of the in vivo tissue sample by determining a scattering phase and a non-scattering phase.

4. The device of claim 3, wherein the scattering phase is associated with the red blood cell volume.

5. The device of claim 4, wherein the non-scattering phase is associated with the intravascular plasma volume.

6. The device of claim 5, wherein the intravascular plasma volume and the red blood cell volume are used to determine the hematocrit level.

7. The device of claim 1, wherein the processor is further programmed to calculate a blood oxygen saturation level of the in vivo tissue sample.

8. The device of claim 7, wherein the blood oxygen saturation level of the in vivo tissue sample is calculated based on the amount of inelastically scattered light collected from collected the in vivo tissue sample.

9. A method of performing in vivo blood and tissue analysis, comprising the steps of:
positioning a light source to illuminate an in vivo tissue sample with at least one pulse of near infrared light;
sending at least one pulse of near infrared light into the in vivo tissue;
collecting with a single channel detector any multi-wavelength light emitted from the in vivo tissue sample in response to the series of pulses of light;
WO 2017/100280 PCT/US2016/065319 using a processor to determine the amount of prompt emission light and the amount delayed emission light in the multi-wavelength light emitted from the in vivo tissue sample.

10. The method of claim 9, further comprising the step of using the processor to calculate an intravascular plasma volume and a red blood cell volume of the in vivo tissue sample based on the amount of prompt emission light and the amount of delayed emission light.

11. The method of claim 10, wherein the step of using the processor to calculate an intravascular plasma volume and a red blood cell volume of the in vivo tissue sample comprises determining a scattering phase and a non-scattering phase.

12. The method of claim 11, wherein the scattering phase is associated with the red blood cell volume.

13. The method of claim 12, wherein the non-scattering phase is associated with the intravascular plasma volume.

14. The method of claim 13, further comprising the step of using the intravascular plasma volume and the red blood cell volume to determine the hematocrit level.

15. The method of claim 9, further comprising the step of using the processor to calculate a blood oxygen saturation level of the in vivo tissue sample.

16. The method of claim 15, wherein the step of using the processor to calculate the blood oxygen saturation level is based on the amount of inelastically scattered light collected from collected the in vivo tissue sample.
